(19) United States
(12) Plant Patent Application Publication
(10) Pub. No.: US 2008/0250536 P1
(43) Pub. Date: Oct. 9, 2008

(54) Cupressus leylandii named 'Madeline'

(76) Inventor: Scott Ahrens, Clermont, FL (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 11/732,220

(22) Filed: Apr. 3, 2007

(51) Int. Cl. A01H 7/00

(52) U.S. Cl. PLT/213

(57) ABSTRACT

A new and distinct cultivar of Cupressus leylandii plant is described herein. The new variety is resistant to fungus and possesses increased growth rate (e.g., height, spread, trunk).

### BOTANICAL DENOMINATION (LATIN NAME)

Cupressus leylandii

### VARIETAL DENOMINATION

‘Madeline’

### BACKGROUND

The Leyland Cypress (Cupressus leylandii) is a fast-growing evergreen tree used in horticulture. They are commonly planted as hedges or screens in gardens as a way to produce a boundary or create more privacy. Although Leyland Cyprus is a quick-growing plant, it has limitations. A major concern of the Leyland Cypress is its susceptibility to disease caused by fungus. For example, in regions with hot summers, Leyland Cyprus is very prone to the cypress canker caused by the fungus Seridium cardinale, which can ultimately kill the tree. Thus, it would be desirable to have a variety of Leyland Cypress that is more resistant to diseases caused by fungus as well as other favorable properties associated with Leyland Cypress. Described herein is a new cultivar of Leyland Cypress that addresses these needs.

### SUMMARY OF THE INVENTION

The new cultivar of Leyland Cypress is a sport of a commercially-available Leyland Cypress grown in a nursery in Newberry, Fla. The new variety of Leyland Cypress named ‘Madeline’ was asexually reproduced in Leesburg, Fla. by rooting cuttings of Leyland Cypress in potted soil. Plants grown from the cuttings were subsequently planted in 5-gallon containers, with some plants grown in Sumterville, Fla. (USDA Hardiness level 9) and others grown in Cairo, Ga. (USDA Hardiness level 8). The plant cannot be reproduced true from seed.

Below is a list of distinguishing features of the new cultivar ‘Madeline’ described herein when grown under normal horticultural practices in Sumterville, Fla. and Cairo, Ga.
1. Resistant to diseases caused by fungus.
2. Increased height growth and spread growth.
3. Dark, attractive green color.
4. Needle tissue is thicker.
5. The needle-like leaves are farther apart from one another.
6. Increased rate of trunk caliper (i.e., thickness).

As shown above, the new cultivar has a number of desirable features. In general, the new cultivar is resistant to fungus infestation when grown in Sumterville, Fla. and Cairo, Ga. This is a desirable feature in view of the susceptibility of commercially-available Leyland Cypress to fungus-causing diseases. The new cultivar also grows faster in height and spread growth by about 30% compared to other varieties of Leyland Cypress. Moreover, rate of trunk growth is faster by about 10% compared to other varieties of Leyland Cypress, which is indicative of a stronger root system typically not present in Leyland Cypress. In summary, the new cultivar is a robust plant that has many desirable features not present in known varieties of Leyland Cypress.

### DESCRIPTION OF THE DRAWINGS

FIG. 1 is a photograph of Cupressus leylandii ‘Madeline’ in a 200 gallon container.

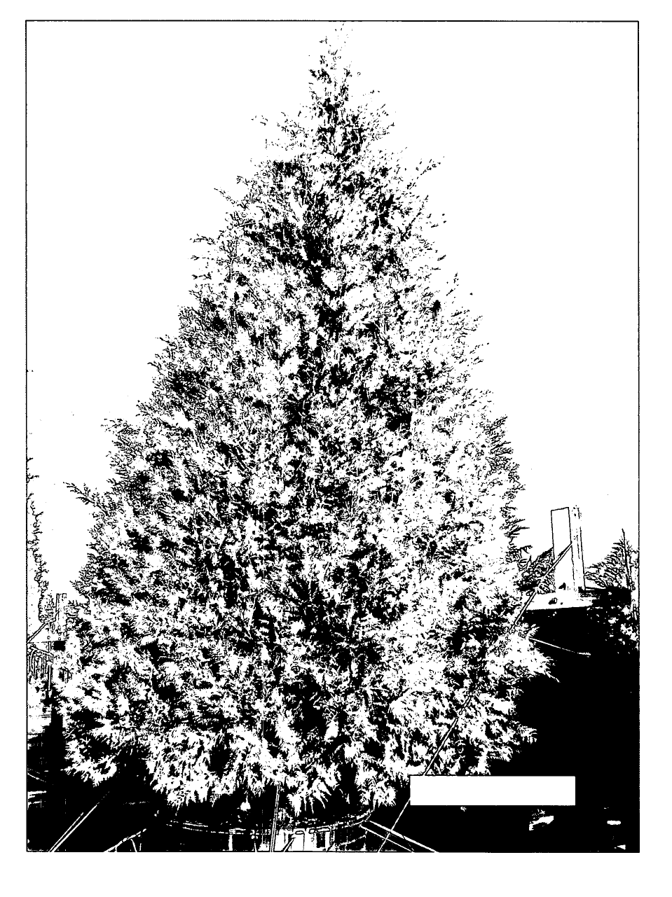

### DETAILED PLANT DESCRIPTION

The following is a detailed description of the new cultivar based on observations of a seven year old specimen grown in a 200 gallon container.
Form: Large, dense, and broadly pyramidal
Height: Approximately 20′
Width: Approximately 9′
Trunk: Diameter between 5¼ and 5½ inches
Needles: Dark green with spacing between needles of at least 0.1 cm farther apart compared to Leyland Cypress
Pests: None serious.

What is claimed:

1. A new and unique variety of Cupressus leylandii plant named ‘Madeline’ as herein shown and described.
